(19) United States
(12) Patent Application Publication
(10) Pub. No.: US 2018/0263860 A1
(43) Pub. Date: Sep. 20, 2018

(54) DELIVERY SYSTEMS FOR APPLICATION OF ANTIBIOTIC GEL FORMULATION

(71) Applicant: Indiana University Research and Technology Corporation, Indianapolis, IN (US)

(72) Inventors: Ghaeth H. Yassen, Indianapolis, IN (US); Jeffrey A. Platt, Indianapolis, IN (US); Richard L. Gregory, Carmel, IN (US)

(21) Appl. No.: 15/756,811

(22) PCT Filed: Aug. 23, 2016
(86) PCT No.: PCT/US16/48165

§ 371 Date: Mar. 1, 2018

Related U.S. Application Data
(60) Provisional application No. 62/214,469, filed on Sep. 4, 2015.

(51) Int. Cl. A61K 6/00

(52) U.S. Cl. CPC A61K 6/0067; A61K 6/002; A61K 6/0035

(57) ABSTRACT

Antibiotic gel formulations for use in dental applications are disclosed. More particularly, the present disclosure is directed to antibiotic gel formulations including low concentrations of antibiotics that are capable of killing root canal pathogens without harming the stem cells inside the root canal. Additionally, the present disclosure is directed to delivery systems and methods for applying the antibiotic gel formulations into a subject's intracanal region.

### CROSS REFERENCE TO RELATED APPLICATIONS

This application claims priority under 35 USC § 119(e) to U.S. Provisional Application Ser. No. 62/214,469 filed on Sep. 4, 2015, the entire disclosure of which is incorporated herein by reference.

### BACKGROUND OF THE DISCLOSURE

The present disclosure relates generally to antibiotic gel formulations for use in dental applications. More particularly, the present disclosure is directed to antibiotic gel formulations including low concentrations of antibiotics that are capable of killing root canal pathogens without harming the stem cells inside the root canal. Additionally, the present disclosure is directed to delivery systems and methods for applying the antibiotic gel formulations into a subject's intracanal region.

Endodontic regeneration procedures are contemporary, biologically based therapies that manage immature teeth with necrotic pulps. These procedures may offer several advantages over traditional treatments of necrotic immature teeth, such as a shorter treatment time and continuous root development. The first critical aspect of endodontic regeneration procedures includes the disinfection of root canal systems using intracanal irrigants, mainly sodium hypochlorite (NaOCl), and medicaments. The most commonly used medicaments during endodontic regeneration are triple antibiotic paste (TAP) and calcium hydroxide (Ca[OH]₂). However, it has been found that conventionally used concentrations of TAP, ranging from above 1 mg/mL, lead to cytotoxic effects against human stem cells of the apical papilla. The use of conventional medicaments may further negatively affect the physical and mechanical properties of radicular dentin, for example, use of these medicaments have been found to reduce dentin flexure strength, microhardness and root resistance to fracture. Furthermore, concerns have been raised regarding the dental discoloration effect of minocycline present in TAP, as well as the development of antimicrobial resistance and an allergic reaction to antibiotic medicaments.

Based on the foregoing, there is a need in the art for antibiotic formulations for use in dental applications such as endodontic regeneration, root canals, and the like. The antibiotic formulations should have antibiotic capability such to effectively kill pathogens within the root canal without harming the stem cells inside the canal. It would be further advantageous if the antibiotic formulations had a paste-like consistency such to maintain its availability within the root canal and improve its application.

### BRIEF DESCRIPTION

In one aspect, the present disclosure is directed to a delivery system for delivering an antibiotic gel formulation into a root canal. The delivery system comprises: an applicator and an antibiotic gel formulation, the antibiotic gel formulation comprising an antibiotic selected from the group consisting of ciprofloxin, metronidazole, and combinations thereof, and a thickening agent.

In another aspect, the present disclosure is directed to a method of applying an antibiotic gel formulation to a root canal. The method comprises: applying the antibiotic gel formulation with a syringe comprising a tip cap and an angled delivery tip shaped to fit into a root canal, the angled delivery tip comprising a diameter of from about 0.25 mm to about 1.25 mm; and allowing the antibiotic formulation to remain in the root canal for a period of from about 1 week to about 2 months.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a syringe for use in the delivery system of one embodiment of the present disclosure.

FIGS. 2A-2C depict the mean (SE) percentage of biofilm formation by E. faecalis treated with various dilutions of formulations and an untreated negative control (set at 100%) at the baseline (immediately after gel formulation preparation) (FIG. 2A), at 1 month after gel formulation preparation (FIG. 2B), and at 3 months after gel formulation preparation (FIG. 2C) as analyzed in Example 1. Within each dilution, different lower case letters indicate statistically significant differences.

FIGS. 3A-3C depict the mean (SE) percentage of biofilm formation by P. gingivalis treated with various dilutions of formulations and an untreated negative control (set at 100%) at the baseline (immediately after gel formulation preparation) (FIG. 3A), at 1 month after gel formulation preparation (FIG. 3B), and at 3 months after gel formulation preparation (FIG. 3C) as analyzed in Example 1. Within each dilution, different lower case letters indicate statistically significant differences.

FIGS. 4A-4C depict scanning electron microscopic images of 3-week old Enterococcus faecalis biofilms under various magnifications showing a thick mat like structure encrusting the entire dentin surface.

FIGS. 5A and 5B depict two different 3D reconstructions of confocal laser scanning microscopy images showing a multilayered structure of 3-week old Enterococcus faecalis biofilms with live and dead (marked with an “X”) cells. Bars represent 50 μm (FIG. 5A) and 70 μm (FIG. 5B).

FIG. 6 is a graph depicting the antibiofilm effects of the different disinfectants represented as the mean of the log CFU/mL as analyzed in Example 2. Different upper case letters indicate a statistical significance.

FIG. 7 is a graph depicting antibiofilm effects of two concentrations of the antimicrobial gels of the present disclosure against clinical isolates obtained from mature and immature teeth as analyzed in Example 3.

FIG. 8 is a graph depicting the residual antibacterial effect of the different concentrations of the antibiotic gel formulations of the present disclosure applied for one or four weeks represented as the mean of the log CFU/mL over time. MC is methylcellulose paste without antibiotic.

FIG. 9 is a graph depicting the residual antibacterial effect of the different concentrations of the antibiotic gel formulations of the present disclosure as the mean of the log CFU/mL against clinical isolates from immature and mature necrotic teeth as analyzed in Examples 5.

FIG. 10A depicts necrotic immature permanent upper incisor with periapical abscess as treated in Example 6.

FIG. 10B depicts the sinus tract of a patient with periapical abscess as treated in Example 6.

FIG. 11 depicts a periapical sinus tract after seven weeks of treatment with the antibiotic gel formulation of the present disclosure as analyzed in Example 6 (2 months follow up).

FIG. 12 depicts a radiograph showing periapical healing as analyzed in Example 6 (6 months follow up).

FIG. 13 depicts a radiograph showing periapical healing as analyzed in Example 6 (one year follow up).

FIG. 14 depicts the absence of any sign of discoloration and inflammation as analyzed in Example 6 (one year follow up).

FIG. 15 is a graph depicting the antibiofilm effects of the different radiopaque antibiotic gel formulations represented as the mean of the log CFU/mL (±SD) as analyzed in Example 7.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

### Antibiotic Gel Formulation

The antibiotic gel formulations of the present disclosure can generally be used in dental applications, and can be used specifically in applications such as endodontic regeneration, root canals, and the like. The antibiotic gel formulations have antibiotic capability such to effectively kill pathogens within the root canal without harming the stem cells inside the canal. Particularly, it has been unexpectedly found that an antibiotic gel formulation including a low concentration of antibiotics can be provided to effectively kill pathogens in the root canal without substantially harming stem cells therein. Further, it has been found that the antibiotic gel formulations of the present disclosure can be easily removed from the root canal, such as by rinsing with ethylenediaminetetraacetic acid (EDTA).

As used herein, “effectively kill pathogens” refers to killing at least 60% of the endodontic pathogens, including at least 70%, including at least 80%, including at least 90%, including at least 95%, including at least 96%, including at least 97%, including at least 98%, including at least 99%, and even including 100% of the endodontic pathogens.

As used herein, “without substantially harming stem cells” refers to killing less than 40% of stem cells, including less than 30% of stem cells, including less than 20%, including less than 15%, including less than 10%, including less than 5%, and including killing 0% of the stem cells inside the root canal.

As used herein, “gel” and “paste” are used interchangeably to refer to a formulation having a viscosity of at least 10,000 centipoise (cps). More suitably, the gel formulations of the present disclosure have a viscosity of from about 10,000 cps to about 50,000 cps.

Generally, the gel formulations include low concentrations of antibiotic. The antibiotic can be ciprofloxin or ciprofloxin in combination with metronidazole. The antibiotic combination of ciprofloxin and metronidazole is commonly referred to in the art as double antibiotic paste (“DAP”). Typically, the antibiotic combination includes equal parts ciprofloxin and metronidazole; that is, the two antibiotics can be included in the formulation in a ratio of about 1:1.

When used alone, ciprofloxin is present in the gel formulations in amounts ranging from about 1 mg/ml to about 50 mg/ml of ciprofloxin, including from about 1 mg/ml to about 30 mg/ml of ciprofloxin, including from about 1.5 mg/ml to about 15 mg/ml, and including from about 2 mg/ml to about 10 mg/ml.

When the combination of ciprofloxin and metronidazole is used, the gel formulations typically include from about 1 mg/ml to about 50 mg/ml of the antibiotic combination, including from about 1 mg/ml to about 30 mg/ml of the antibiotic combination, including from about 1.5 mg/ml to about 15 mg/ml, and including from about 2 mg/ml to about 10 mg/ml. It has been found that the use of about 10 mg/ml of the antibiotic combination in the formulation of the present disclosure kills from about 10% to about 20% stem cells. It should be understood by one skilled in the art that the tolerance for not killing stem cells is stricter during the early childhood years. For example, the presence of stem cells in children aged 8 years to 16 years is greater and more substantial than in a subject older than 16 years. Accordingly, in some embodiments, the formulation for administration to a child subject should be prepared to include a lower concentration of the antibiotic combination than that of a formulation administered to an adult subject (i.e., subject over 16 years of age) such to further limit the harm to stem cells.

The low concentrations of antibiotics in the antibiotic gel formulations of the present disclosure provide the formulations with a neutral pH; that is, a pH of from about 6 to about 7. This is surprising as typically antibiotic gel formulations are acidic in nature, and this acidic nature is helpful in maintaining chemical stability and physiological compatibility. However, as shown in the Examples below, the low concentration antibiotic gel formulations having a neutral pH have been unexpectedly found to be chemically and physiologically stable for up to three months after application of the formulation. In particularly suitable embodiments, the antibiotic gel formulations will be retained into the root canal for a period of from about 1 week to about 2 months, including from about 2 weeks to about 4 weeks prior to removal, and thus, the formulations should remain chemically stable and physiological compatible for at least those periods of time.

The formulations of the present disclosure may further include a thickening agent to provide a paste-like consistency for the formulation to maintain its availability inside the root canal and improve its application. That is, the viscosity of the formulation should be such that it can be easily pumped into the root canal, but can also be retained within the canal once applied. In one particularly suitable aspect, the thickening agent is methylcellulose. Methylcellulose is considered to increase the duration of therapeutic drug release, thus prolonging the effects. Furthermore, the non-cytotoxic nature of methylcellulose makes it one of the most commonly used culture media for stem cell growth and differentiation, which is helpful during endodontic regeneration procedures. Other suitable thickening agents include, for example, propylene glycol, polyethylene glycol (e.g., macrogol), and combinations thereof.

Typically, the formulations of the present disclosure include from about 60 mg/ml to about 110 mg/ml thickening agent, including from about 70 mg/ml to about 80 mg/ml thickening agent, and from about 90 mg/ml to about 100 mg/ml thickening agent.

In some aspects, the formulations should further include an imaging agent (as referred to herein as “radiopaque material”). For example, the formulation may include a radiopaque material such that the formulation can be visible during and after application using dental radiograph. In one particularly suitable embodiment, the radiopaque material is a barium-containing material, and in a particularly suitable embodiment, is barium sulfate. Other suitable imaging agents include, for example, bismuth oxide, zirconium oxide, titanium oxide, lothalamate meglumine, and combinations thereof.

Typically, when present, the antibiotic gel formulations will include an imaging agent in amounts of from about 0.15 g/ml to about 0.40 g/ml and including from about 0.25 g/ml to about 0.35 g/ml.

It should be further understood that in embodiments where the formulations include an imaging agent, higher concentrations of the antibiotic combination may be required. Typically, when an imaging agent is included, the antibiotic gel formulations include from about 5 mg/ml to about 30 mg/ml antibiotic.

In some aspects, an effective amount of the antibiotic combination in the formulations described herein may be further mixed with one or more excipients or diluted by one or more excipients. Excipients may serve as a diluent, and can be solid, semi-solid, or liquid materials, which act as a vehicle, carrier, preservative or medium for the active ingredient. Thus, the antibiotic gel formulations may contain anywhere from about 0.1% by weight to about 20% by weight active ingredients (i.e., antibiotic combination), depending upon the selected dose and dosage form.

### Preparing the Antibiotic Gel Formulation

In general, the methods for preparing the antibiotic gel formulation of the present disclosure include: dispersing an antibiotic in water to form an antibiotic solution; and mixing a thickening agent with the antibiotic. In one embodiment, the antibiotic is ciprofloxacin. In another embodiment, the antibiotic is an antibiotic combination prepared by mixing amounts of metronidazole and ciprofloxacin. As noted above, in one suitable embodiment, the metronidazole and ciprofloxacin are mixed to form an antibiotic combination including equal parts ciprofloxin and metronidazole.

When used alone, ciprofloxin is dispersed in water such to provide an antibiotic gel formulation including from about 1 mg/ml to about 50 mg/ml of ciprofloxin, including from about 1 mg/ml to about 30 mg/ml of ciprofloxin, including from about 1.5 mg/ml to about 15 mg/ml, and including from about 2 mg/ml to about 10 mg/ml.

When ciprofloxin is combined with metronidazole, once the antibiotics are mixed, the antibiotic combination is dispersed in water. Typically, sufficient amounts of each of metronidazole and ciprofloxacin are mixed and dispersed in amounts of water such to provide an antibiotic gel formulation including from about 1 mg/ml to about 50 mg/ml of the antibiotic combination, including from about 1 mg/ml to about 30 mg/ml of the antibiotic combination, including from about 1.5 mg/ml to about 15 mg/ml, and including from about 2 mg/ml to about 10 mg/ml.

Thickening agents and amounts of thickening agents for use in the methods include those discussed above. In suitable aspects, the thickening agent is mixed with the antibiotic solution to form a gel formulation including from about 60 mg/ml to about 110 mg/ml thickening agent, including from about 70 mg/ml to about 80 mg/ml thickening agent, and including from about 90 mg/ml to about 100 mg/ml thickening agent.

Suitably, in some aspects, the thickening agent is mixed with the antibiotic solution intermittently such to slowly add a portion of the thickening agent to the antibiotic solution at a time. For example, in one embodiment, about ¼ of the thickening agent is added from about every 10 minutes to about every 15 minutes until all thickening agent is added with the antibiotic solution.

In some aspects, once completely added, the thickening agent is mixed with the antibiotic solution for an additional period of from about 1 hour to about 2 hours to ensure that the prepared antibiotic gel formulation has a homogenous pasty consistency with a viscosity of from about 10,000 cps to about 50,000 cps.

In some aspects, an imaging agent is further mixed with the antibiotic solution in the methods of the present disclosure. For example, a radiopaque material is mixed with the antibiotic solution such that the resulting gel formulation can be visible during and after application using dental radiograph. In one particularly suitable embodiment, the radiopaque material/imaging agent is a barium-containing material, and in a particularly suitable embodiment, is barium sulfate. Other suitable imaging agents include, for example, bismuth oxide, zirconium oxide, titanium oxide, lothalamate meglumine, and combinations thereof.

Typically, when mixed with the antibiotic solution, the resulting antibiotic gel formulations will include an imaging agent in amounts of from about 0.15 g/ml to about 0.4 g/ml and including from about 0.25 g/ml to about 0.35 g/ml.

Further, in some aspects, the methods of the present disclosure provide for storing the antibiotic gel formulation for at least 24 hours prior to use.

### Delivery System for Application of Antibiotic Gel Formulation

In another aspect, the present disclosure is directed to a delivery system including an applicator for application of the antibiotic gel formulation. It should be understood that although described herein with the use of a syringe as the applicator, the antibiotic gel formulation may be used with any applicator capable of introducing the formulation into the root canal as known in the art without departing from the present disclosure.

Further, the antibiotic gel formulation described herein may be administered in a single dose or in multiple doses over a time period. In some clinical situations, one or two additional doses of the antibiotic gel can be applied every 1-4 weeks in order to control the local infection. The dose should be given only during the dental procedure and should be administered by a dental professional. Further, in some suitable embodiments, the antibiotic gel formulations will be retained into the root canal for a period of from about 1 week to about 2 months, including from about 2 weeks to about 4 weeks, and thus, the formulations should remain chemically stable and physiological compatible for at least those periods of time.

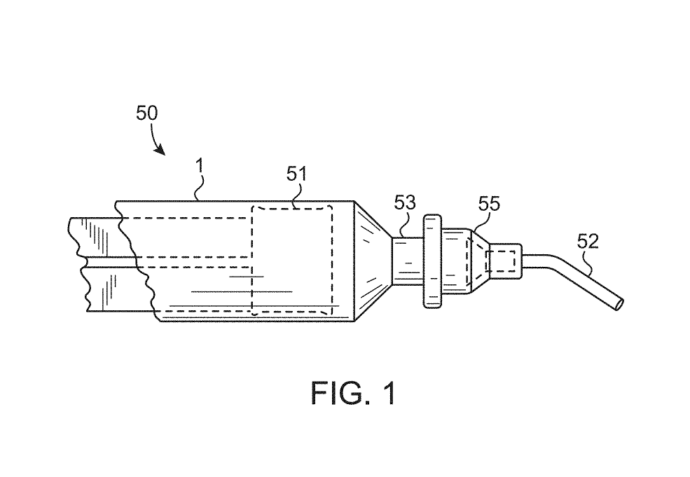

In one particularly suitably embodiment, the delivery system includes the above described antibiotic gel formulation administered using a syringe (see FIG. 1). Typically, the syringe 50 includes a standard tubular design. It is particularly suitable that the tubular member 1 of the syringe 50 be made of a non-reactive clear or dark plastic to enable the operator of the syringe to visually monitor the amount of formulation within the tubular member 1. The tubular member 1 is fitted with a plunger 51 slidably received therein so that the inside walls of the tube and the outer edge of the plunger 51 produce a tight fit around the circumference of the plunger 51.

Typically, the total volume of the syringe is from about 0.5 ml to about 2.0 ml and including from about 0.8 ml to about 1.4 ml. Further, the syringe has a diameter ranging from about 3 mm to about 5 mm and including about 4 mm.

A syringe tip cap 55 is then screwed onto the luer 53 of the syringe 50. The male luer lock of the syringe securely mates with the female luer lock of the syringe tip cap 55. Alternatively, the connection can be secured by a friction fit between the outer circumference of the syringe tip and the inner circumference of the cap. Once the syringe tip cap is set securely onto the syringe luer 53, an airtight fit is obtained.

The syringe tip cap 55 includes a delivery tip 52 shaped to fit the end of the delivery tip 52 facing away from the tubular member 1 of the syringe 50 into the root canal (not shown). While shown as an angled delivery tip 52, it should be understood by one skilled in the art that the delivery tip 52 can be straight, non-angled without departing from the scope of the invention. Typically, the delivery tip 52 has a length of from about 10 mm to about 30 mm, including from about 15 mm to about 25 mm, and including about 20 mm. Further, as the formulation to be applied using the applicator has a paste-like consistency, it should be appreciated that the diameter of the delivery tip should be such to allow the formulation to freely flow therethrough. Typically, the diameter of delivery tip ranges from about 0.25 mm to about 1.25 mm, including from about 0.5 mm to about 1.0 mm, and including from about 60 mm to about 80 mm.

In some embodiments, the tip cap further includes a stopper (not shown) that prevents the clinician from positioning the delivery tip too deep within the root canal. Depending on the length of the root canal, the delivery tip can move into the root canal from about 3 mm to about 15 mm and this can be adjusted by the stopper. Typically, the stopper is comprised of plastic and is used as a reference point to control the length of the delivery tip during insertion into the root canal and injection of the antibiotic gel formulation.

Typically, the delivery system including the applicator and antibiotic gel formulation can be used to disinfect 2-5 root canals, depending on the type of tooth, length of tooth and internal diameter of the root canal.

The following examples further illustrate specific embodiments of the present disclosure; however, the following illustrative examples should not be interpreted in any way to limit the disclosure.

### EXAMPLES

### Example 1

In this Example, the antibiotic gel formulation of the present disclosure was made and analyzed for its inhibitory effect against biofilm formation by Enterococcus faecalis and Porphyromonas gingivalis. This inhibitory effect was compared to the inhibitory effect of a modified triple antibiotic paste (MTAP).

### Materials and Methods

Formulation Preparation and Loading with DAP and MTAP

Antibiotic formulations were prepared as follows: to prepare 1 mg/mL methylcellulose-based MTAP, 50 mg of United States Pharmacopeia grade antibiotic powders compounded of 43% clindamycin, 14% ciprofloxacin, and 43% metronidazole (Skywalk Pharmacy, Wauwatosa, Wis., USA) was dissolved in 50 mL of sterile water. Then, 4 grams of methylcellulose powder (Methocel 60 HG, Sigma-Aldrich, St Louis, Mo., USA) was added to the mixture and stirred for 2 hours at room temperature to obtain a homogeneous antibiotic gel formulation. The gel was left to stand for an additional 2 hours to ensure the complete disappearance of all foam from the mixture. To prepare 1 mg/mL methylcellulose-based antibiotic gel formulation of the present disclosure (DAP), 50 mg of United States Pharmacopeia grade antibiotic powders compounded of equal portions of metronidazole and ciprofloxacin (Champs Medical, San Antonio, Tex., USA) were used, and the antibiotic gel formulation was prepared as described above. An antibiotic-free placebo formulation composed of sterile water and methylcellulose was also prepared utilizing the same method.

The viscosity of the prepared formulations was selected based on pilot studies that had examined the viscosities of various methylcellulose-based formulations. A formulation viscosity that had sufficient consistency to be used as an intracanal medicament and applied to root canals using commercially available endodontic syringe tips (NaviTips, Ultradent, South Jordan, Utah, USA) was selected. The pH of the prepared formulations was measured in triplicate during this Example, and the values for DAP, MTAP, and placebo gels were 7.2, 7.6, and 7.7, respectively.

Bacterial Strains and Culture Conditions

Enterococcus faecalis (ATCC 29212) and Porphyromonas gingivalis (ATCC 33277) strains were used in this Example. E. faecalis and P. gingivalis were selected as representative common endodontic pathogens that are present in various types of endodontic infections. E. faecalis is a gram-positive facultative anaerobe that has been detected in 67-77% of cases of secondary root canal infection. On the other hand, P. gingivalis is a gram-negative obligatory anaerobe that has been detected in 44-48% of cases of primary root canal infection. Each bacterial strain was initially grown on anaerobic blood agar plates (CDC, BioMerieux, Durham, N.C., USA), and then grown and maintained as described in Sabrah A H, et al., (2013) J Endod 39, 1385-1389 utilizing sterile Brain Heart Infusion broth supplemented with 5 grams of yeast extract/L (BHI-YE; Becton Dickinson Co., Franklin Lakes, N.J., USA) containing 5% v/v vitamin K (0.5 mg/mL) and hemin (50 mg/mL) (Remel, Lenexa, Kans., USA). Both test bacteria were grown in an anaerobic environment created using gas-generating sachets (Gas-Pak EZ; Becton) and incubated for 48 hours in an incubator at 37° C. Bacterial growth was confirmed by changes in turbidity at 48 hours. The number of colony-forming units/mL for E. faecalis and P. gingivalis after 48 hours was 1.78×10⁸ (optical dentistry=0.6 at 600 nm) and 3.6×10⁸ (optical dentistry=0.67 at 600 nm), respectively.

Determination of Biofilm Inhibition

The ability of the prepared gels to inhibit biofilm formation by E. faecalis and P. gingivalis was tested as described previously in Sabrah A H, et al. (2013) J Endod 39, 1385-1389. In summary, 250 μL of two-day-old cultures of E. faecalis and P. gingivalis broth were treated with 5 mL of 1:10, 1:20, 1:40, 1:80, and 1:160 dilutions of freshly prepared MTAP, DAP, or placebo formulations in BHI-YE. Inoculated E. faecalis and P. gingivalis broth without formulations served as negative controls. The treated and untreated bacterial media were incubated anaerobically at 37° C. for 48 hours in 96-well microtiter plates (200 μL per well). The culture fluid was carefully withdrawn without touching the formed biofilms using a multichannel pipette to remove planktonic bacteria. Then, the biofilm in each well was gently washed twice with sterile 0.9% saline, fixed for 30 minutes with 10% formaldehyde, washed two additional times with 0.9% sterile saline, and stained for 30 minutes with 0.5% crystal violet. The biofilm in each well was washed three more times with sterile 0.9% saline to remove any unbound crystal violet, and the crystal violet bound to the biofilm was then extracted by adding 200 μL of 2-propanol for 1 hour. The extract was diluted 1:5 with 2-propanol and the optical absorbance was measured at 490 nm using a microplate spectrophotometer (Spec-traMax 190; Molecular Devices, Sunnyvale, Calif., USA). 2-Propanol was used as a blank control. The same batches of the prepared formulations were stored at 4° C. and the microtiter plate antibiofilm test was repeated after the formulations had been aged for one and three months to verify the antibacterial stability of the prepared gels over time.

Statistical Analysis

Each experiment was conducted two separate times using two independent batches of the prepared formulations, and three readings were obtained for each dilution in each experiment. The percentage of biofilm formation after various treatments was calculated using the equation:

Biofilm formation (%)=(experimental absorbance value)/(untreated negative control absorbance value)×100.

The percentages of biofilm formation in the presence or absence of the treatment gels were analyzed statistically using a mixed-model ANOVA followed by Fisher's least significant difference test for pairwise comparisons. A random effect to correlate the data within each experiment was also included. The significance level was set at 0.05.

### Results

Antibiofilm Effect Against E. faecalis

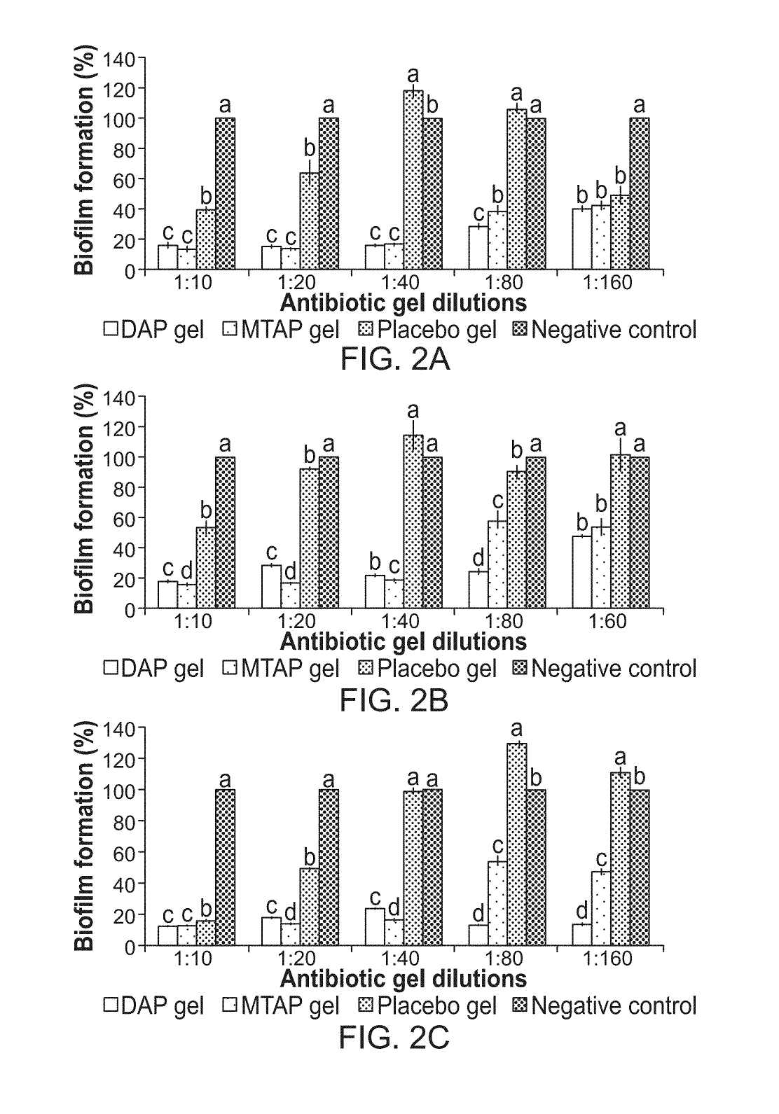

FIGS. 2A-2C indicate that the MTAP and DAP formulations caused significant reduction of biofilm formation relative to the untreated negative control bacteria at all tested dilutions through all time points (P<0.00001). Furthermore, both the DAP and MTAP formulations significantly reduced biofilm formation relative to the placebo formulation at all dilutions regardless of the tested time points (P<0.0001) except for 1:160 dilution of the gels tested at the baseline. At the baseline (FIG. 2A), there was no significant difference between the MTAP and DAP formulations at any of the tested dilutions, except for 1:80 where DAP demonstrated significantly higher biofilm-inhibitory activity than did MTAP (P=0.031). After 1 and 3 months (FIGS. 2B and 2C), the MTAP formulation provided a significantly higher biofilm-inhibitory effect than the DAP formulation at the majority of lower dilutions over the range 1:10-1:40 (P=0.01-P<0.00001). On the other hand, the DAP formulation exhibited a significantly stronger biofilm-inhibitory effect than did the MTAP formulation at dilutions of 1:80 and 1:160 (P<0.00001). The placebo gel demonstrated a significant biofilm-inhibitory effect at lower dilutions (1:10, 1:20) relative to the negative control (P<0.001-P<0.00001), regardless of the time point at which the formulations were tested. However, no significant differences were found between the placebo formulation and the negative control at the majority of higher dilutions over the range 1:40-1:160 through all of the tested time points. Furthermore, the placebo formulation allowed significantly higher degrees of biofilm formation relative to the negative control at the baseline for the 1:40 dilution (P=0.0002), as well as at three months for the 1:80 and 1:160 dilutions (P<0.0001 and P=0.0011, respectively).

Antibiofilm Effect Against P. gingivalis

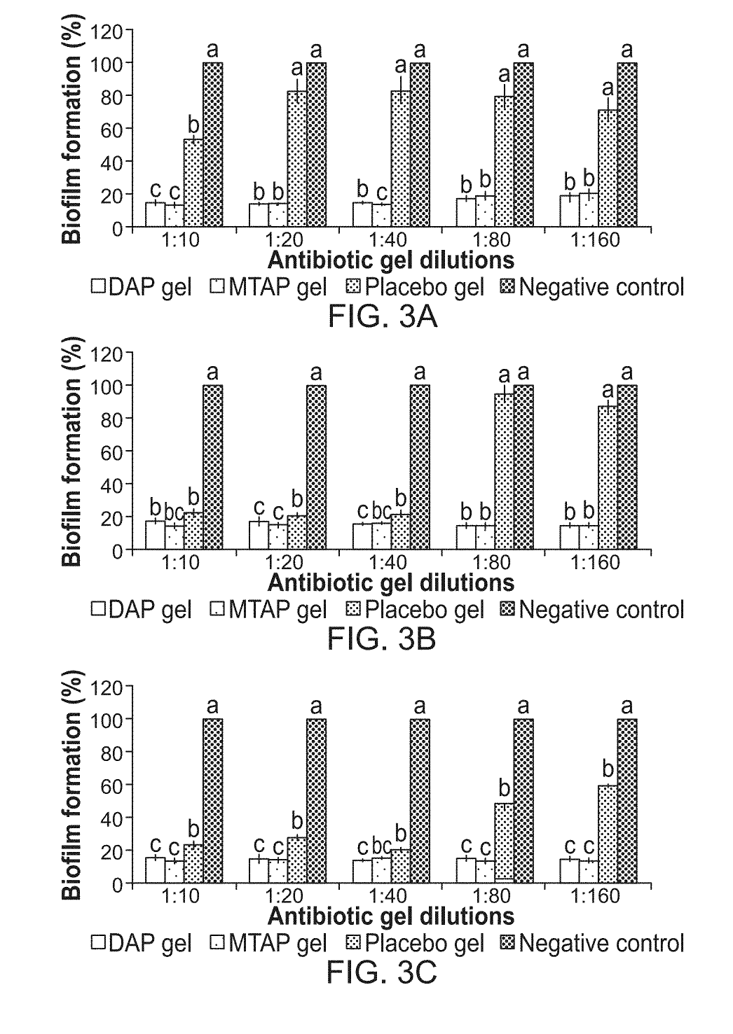

FIGS. 3A-3C show that the MTAP and DAP formulations reduced biofilm formation significantly relative to the untreated negative control bacteria at all time points, regardless of the tested dilution (P<0.00001). Additionally, both the DAP and MTAP formulations significantly reduced biofilm formation relative to the placebo formulation at all dilutions through all time points (P=0.042-P<0.00001) except for the 1:10 dilution of DAP formulation at one month, the 1-month MTAP formulation at a 1:40 dilution and the 3-month MTAP formulation at 1:40 dilutions. No significant differences in biofilm formation were observed between the MTAP and DAP formulations at all time points, regardless of the tested dilutions, except for the baseline 1:40 dilution, where MTAP demonstrated a significantly stronger biofilm-inhibitory effect than DAP (P=0.007). At the baseline (FIG. 3A), the placebo formulation provided no significant reduction of biofilm formation relative to the untreated negative control bacteria at all dilutions except for 1:10 (P<0.0006). One month after gel preparation, the placebo formulation demonstrated a significant reduction in biofilm formation relative to the untreated negative control bacteria at all dilutions (P<0.0001) except 1:80 and 1:160 (FIG. 3B). Three months after preparation of the gel (FIG. 3C), the placebo formulation exhibited a significant reduction in biofilm formation relative to the untreated negative control bacteria at all dilutions (P=0.027-P<0.0001).

### Discussion

The results indicate that methylcelluose-based DAP and MTAP formulations significantly reduced biofilm formation by both species of tested bacteria at all dilutions, regardless of the length of formulation aging time. Additionally, the DAP and MTAP formulations demonstrated significant reduction of biofilm formation by both of the tested bacterial species relative to the placebo formulation at the vast majority of tested dilutions at all of the tested time points. Furthermore, it was found that various dilutions of both antibiotic gel formulations (1:10-1:160) reduced biofilm formation significantly.

Furthermore, it is worth mentioning that 1 mg/mL methylcelluose-based MTAP reportedly has minimal adverse effects on the microhardness and chemical structure of radicular dentin in comparison with the concentration of 1 g/mL used clinically.

The present Example demonstrated that the placebo methylcellulose formulation provided a significant reduction of biofilm formation relative to the negative control at some of the tested dilutions, primarily the lower dilutions. The viscous nature of the prepared formulations, including the placebo, may interfere with bacterial attachment and affect biofilm formation at low dilutions in the microtiter plate model used in this Example. Furthermore, various placebo formulations (vehicles) have been shown to exert significant antibacterial effects against P. gingivalis.

Various substrates, such as dentin, polystyrene microtiter plates, hydroxyapatite disks, and nitrocellulose membrane filters, can be used to determine the anti-biofilm effects of endodontic materials. A crystal violet biofilm assay using polystyrene microtiter plates, which has been widely reported in the endodontic literature was used in the present Example. This is a standardized assay that allows rapid retrieval and quantification of bacterial biofilms. However, the use of a dentin substrate for biofilm formation is more representative of the actual clinical situation. Therefore, the antibiofilm effect of the antibiotic gel formulations tested in this Example will need to be confirmed using a dentin biofilm model.

Within the limitations of this in vitro Example, it appears that DAP and MTAP formulations at 1 mg/mL facilitate significant reduction of biofilm formation by E. faecalis and P. gingivalis at all tested dilutions, even after aging of the formulation preparations for one and three months. These antibiotic gel formulations can be considered as potential intracanal medicaments during endodontic regeneration procedures.

### Example 2

In this Example, the antibiofilm effect of various disinfectants used in endodontic regeneration including irrigation solutions, clinically used intracanal medicaments, and the antibiotic gel formulations of the present disclosure loaded into a vehicle system (e.g., methylcellulose) were analyzed.

### Materials and Methods

Dentin Sample Preparation

Unidentified intact human teeth were collected, stored in 0.1% thymol solution at 4° C., and used within 6 months after obtaining local Institutional Review Board approval (IRB #1409251353). A standardized radicular dentin sample (4×4×1 mm³) was obtained from each root using a slow diamond saw (IsoMet, Buehler, Lake Bluff, Ill.) under continuous distilled water irrigation. The pulpal side of each specimen was wet-finished with silicon carbide abrasive papers (500-2400 grit, Struers, Cleveland, Ohio). The polished samples were then sonicated in deionized water for 3 minutes, washed with sterile water, wrapped individually with moist cotton pellets, placed in Whirl-pak bags (Sigma-Aldrich, St Louis, Mo.), gas sterilized with ethylene oxide, and stored at 4° C. until used.

Bacterial Strain and Media

E. faecalis (ATCC 29212) was grown initially on anaerobic blood agar plates (CDC, BioMerieux, Durham, N.C.). Colonies of E. faecalis were then suspended in brain-heart infusion (BHI) broth supplemented with 5 grams yeast extract/L (BHI-YE) and incubated for 24 hours at 37° C. with 5% CO₂.

Biofilm Growth on Dentin Specimens

Dentin samples were placed individually in separate wells of a 96-sterile well plate (Fisherbrand, Fischer Scientific) with the pulpal side facing upwards. Then, 10 μl of an overnight E. faecalis culture (10⁶ CFU/mL) dispersed in 190 μL of fresh BHI-YE growth media was added to each dentin specimen. Dentin samples were incubated anaerobically for three weeks at 37° C. and the growth medium was replenished every other day.

Biofilm Characterization

The three-week old bacterial biofilm grown on dentin samples was characterized for viability, thickness, homogeneity, and presence of extracellular polymeric substance (EPS) using scanning electron microscopy (SEM; JEOL 7800F, Peabody, Mass.) and confocal laser scanning microscopy (CLSM; FV1000, Olympus Corp, Center Valley, Pa.). For SEM characterization, infected dentin samples (n=2) were washed with PBS to remove unattached bacteria and fixed with 2% glutaraldehyde and 2% paraformaldehyde in phosphate buffer. Ascending concentrations of ethyl alcohol in hexamethyldisilazane (Electron Microscopy Sciences, Fort Washington, Pa.) were used for chemical dehydration. Samples were sputter coated and images were taken with SEM at various magnifications in secondary electron imaging mode. For CLSM characterization, the bacterial biofilms on dentin samples (n=2), were stained with equal volumes of Live and Dead Baclight dye (Molecular Probes, Eugene, Oreg.) according to the manufacturer's instructions, incubated in the dark for 15 minutes and viewed under CLSM. Three randomly selected biofilm areas were scanned from each infected dentin sample and viewed using Olympus FV10-ASW software (Olympus Corp., USA). Live/dead quantification and three-dimensional analysis were performed using Imaris software (version 7.7, Bitplane, South Windsor, Conn.).

Disinfectant Preparation

A total of six disinfectants were tested in this Example including four intracanal medicaments and two irrigation solutions. A commercially available Ca(OH)₂ (UltraCal XS; Ultradent, South Jordan, Utah) was used as well as a clinically used concentration of DAP (500 mg/mL), which was prepared by mixing 500 mg of equal portions of metronidazole and ciprofloxacin USP grade powders (Champs Pharmacy, San Antonio, Tex.) with 1 mL of sterile water. Low concentrations of DAP (1 and 0.1 mg/mL) were also used after loading into a vehicle system to create a pasty consistency that can be applied clinically using commercial application tips (NaviTip, Ultradent). The preparation of the antibiotic gel formulations of the present disclosure was as follows: 100 and 10 mg of DAP powders were dissolved in 100 mL of sterile water, respectively. Then, 8 grams of methylcellulose powder (Methocel 60 HG, Sigma-Aldrich) was gradually incorporated into each diluted DAP solution under vigorous stirring at room temperature to obtain homogenous paste formulations with 1 and 0.1 mg/mL concentrations of DAP. A placebo methylcellulose paste with no DAP was also prepared. For the irrigants used, 1.5% NaOCl and 2% CHX were freshly prepared by diluting 3% and 20% stock solutions of NaOCl (Value Bleach, Kroger, Cincinnati, Ohio) and CHX (Sigma-Aldrich) in sterile water, respectively.

Treatment of Infected Dentin Samples

After three weeks of incubation, the infected dentin samples were randomized into four intracanal medicament treatment groups, two irrigation treatment groups, and two control groups (n=8 per group). For the medicament groups, samples were transferred into individual wells of 48-well plates containing 100 μl of BHI-YE growth media (Corning Life Sciences, Tewksbury, Mass.). The pulpal sides (biofilm growth sides) were treated with 50 μL of one of the three concentrations of DAP (500, 1, or 0.1 mg/mL) or Ca(OH)₂. Samples were stored for seven days at 37° C. and 100% humidity. The same experimental setting was also used to treat the two control groups with sterile saline or placebo paste (methylcellulose only) for seven days. For the irrigation groups, each dentin sample was immersed in 1 mL of sterile saline for 1 minute to remove loosely attached planktonic bacteria followed by immersion in 1 mL of 1.5% NaOCl or 2% CHX for 5 minutes.

Biofilm Disruption Assay

After the assigned treatments, the samples were gently washed for 1 minute with 5 mL of sterile saline to remove the medicaments or remnants of irrigation solutions. Biofilm disruption assays were then performed as described in Sabrah A H, et al. (2015). J Endod 41:1081-4. In summary, each specimen was immersed in a sterile plastic test tube containing 1 mL of sterile saline, vortexed for 10 seconds, sonicated for 10 seconds, and then vortexed again for 10 seconds to detach biofilm cells. A pilot study was conducted to confirm that the used biofilm detachment technique did not cause any bacterial lysis and false negative results. The separated biofilms were then diluted, spiral plated on blood agar plates, and incubated for 24 hours in 5% CO₂ at 37° C. The CFUs/mL were quantified using an automated colony counter (Synbiosis, Inc., Frederick, Md.).

Statistical Analyses

Summary statistics were calculated for the log-transformed data from each group. Because the data is not normally distributed and groups have heterogeneous variances, re-sampling-based permutation tests followed by Sidak post hoc multiple comparison (α=0.05) were used to compare the antibiofilm effect of various experimental groups.

### Results

Biofilm Validation

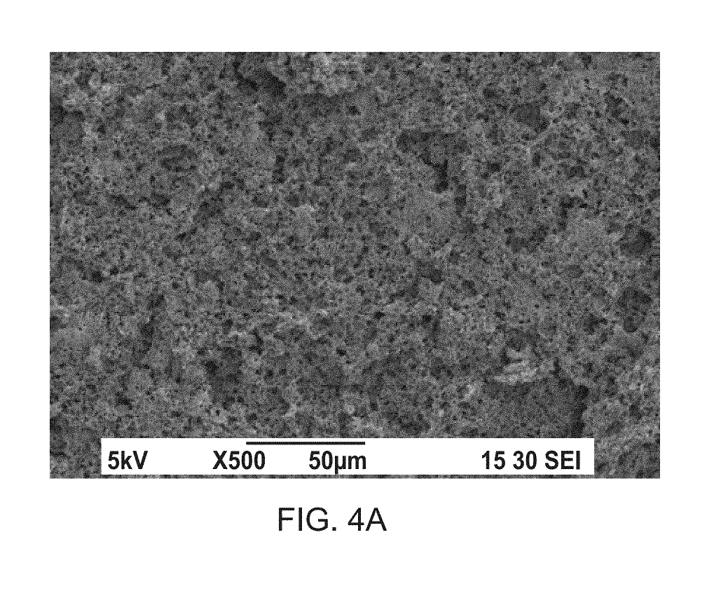

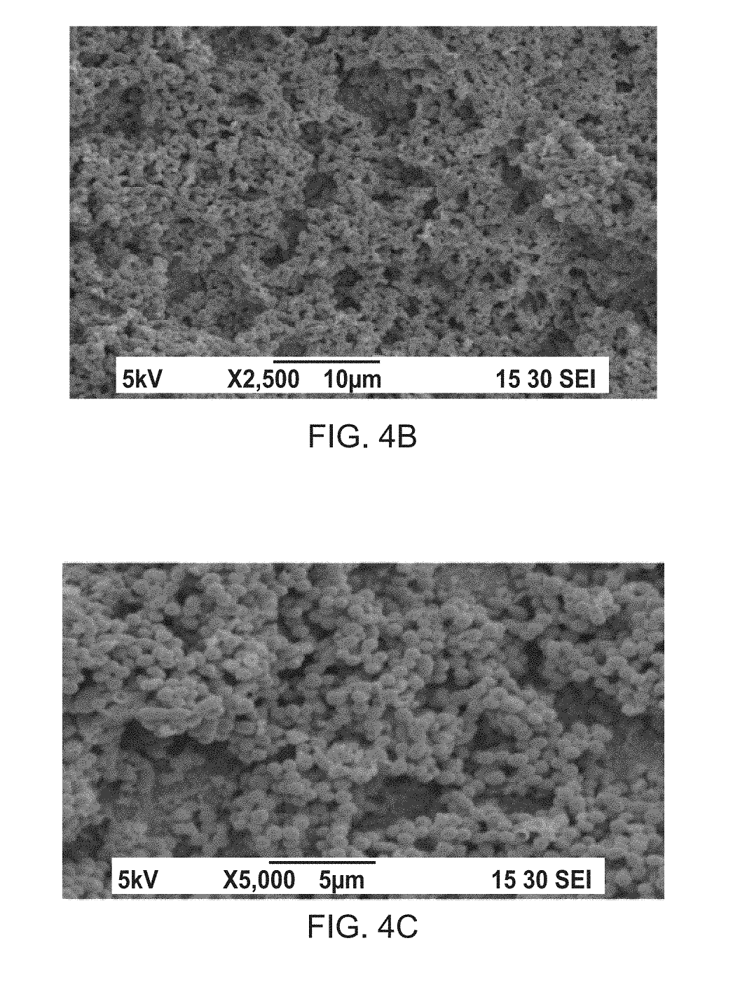

The SEM images demonstrated a thick, uniform mat like biofilm structure covering the whole dentin surface (FIGS. 4A-4C). Interconnected EPS matrix was also observed under higher magnifications. In addition, CLSM exhibited multi-layered three-dimensional biofilm structure covering dentin surface and containing both live and dead (marked with an “X”) bacteria (FIGS. 5A and 5B). The percentage of live cells in the biofilm was 78±5 and the biofilm thickness was 35±5 μm.

Antibiofilm Effect of Disinfectants

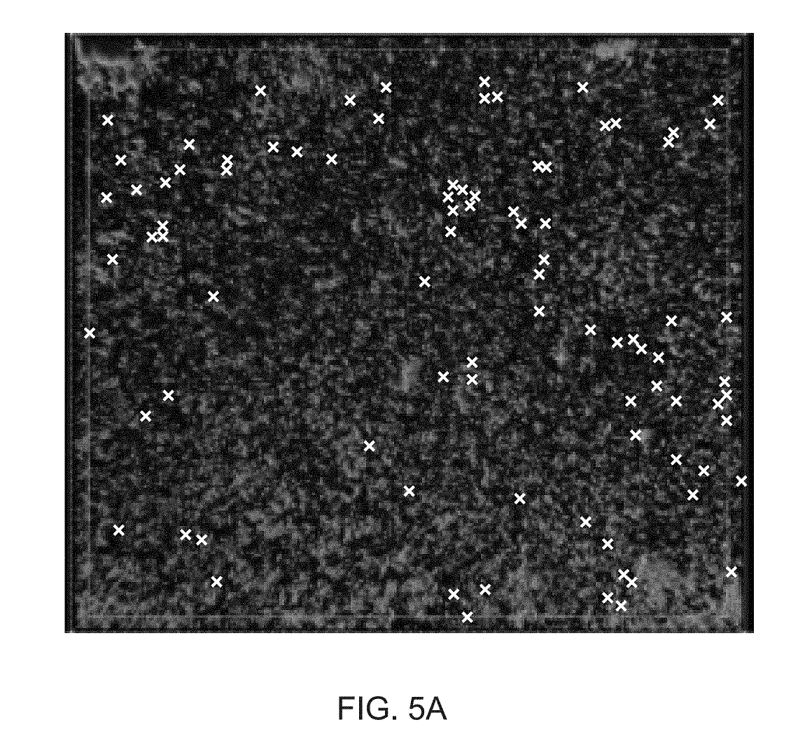

FIG. 6 indicates that infected dentin treated with 1.5% NaOCl or 500 mg/mL DAP had significant reduction and complete eradication of E. faecalis biofilm in comparison to 1 mg/mL of DAP (P=0.02), 0.1 mg/mL DAP (P=0.01) and the control groups (P=0.01). Furthermore, infected dentin treated with 2% CHX, Ca(OH)₂, or 1 mg/mL DAP had significant reduction in E. faecalis biofilm in comparison to both control groups (P=0.01) but were not able to completely eradicate E. faecalis biofilm. Additionally, infected dentin samples treated with 0.1 mg/mL DAP provided significant but limited reduction in E. faecalis biofilm in comparison to the control groups (P=0.02).

### Discussion

The significant biofilm inhibitory effects of low concentrations of DAP in comparison to placebo paste reported in this Example indicates that the antibiofilm properties can be attributed to the active antibiotic ingredients rather than to the methylcellulose vehicle system.

The current clinical recommendation of the American Association of Endodontists suggests the use of 0.1 mg/mL of antibiotic mixtures as inter-appointment disinfectant during endodontic regeneration. However, this Example demonstrated that 0.1 mg/mL of DAP had significant but limited antibiofilm effect. The limited antibiofilm effect of 0.1 mg/mL of DAP could be justified by the well-established three-week old biofilm used in the current Example, which was found to be more resistant to endodontic disinfectants compared to younger biofilms. On the other hand, 1 mg/mL of DAP provided significant antibiofilm effect, eliminated the majority of E. faecalis biofilm, and caused more than a 3 log₁₀ reduction in CFU/mL (more than 99.9% decrease in viable bacteria). Recent reports indicated no cytotoxic effect of 1 mg/mL of DAP against stem cells from apical papillae and dental pulp stem cells. Therefore, 1 mg/mL of DAP may be used as a stem cell friendly inter-appointment disinfectant during endodontic regeneration. Ca(OH)₂ and 500 mg/mL DAP demonstrated significant antibiofilm effect and eradicated most or all of the bacterial biofilm, respectively. However, such a high concentration of DAP was found to be toxic to various stem cells and had a negative effect on both the mechanical properties and chemical structure of the root dentin.

Ca(OH)₂ was suggested to have no deleterious effect on stem cells from apical papillae. However, Ca(OH)₂ can adversely affect the mechanical, physical, and chemical properties of surface dentin within few weeks. Additionally, endodontic regeneration cases disinfected with Ca(OH)₂ were suggested to have less favorable clinical outcomes compared to cases treated with antibiotic medicaments.

In this Example, 1.5% NaOCl provided complete eradication of E. faecalis biofilm. The current Example indicated that 2% CHX was able to nearly eliminate the bacterial biofilm. Although 2% CHX solution has been efficiently used alone or in combination with other irrigants for disinfection during regenerative endodontics, recent in vitro studies suggested highly unfavorable effects of 2% CHX on survival and attachment of stem cells from apical and dental pulp, respectively. Furthermore, it is well documented that CHX is more effective on gram-positive than on gram-negative bacteria. Therefore, the potent antibiofilm effect of CHX observed in this Example against E. faecalis, a gram positive species, may represent an overestimation of the clinical efficiency of this solution.

The antibiofilm effects of irrigation solutions examined in this Example were comparable to that of the clinically used intracanal medicaments (500 mg/mL DAP and Ca(OH)₂). This indicates that the use of a comprehensive irrigation protocol during endodontic regeneration procedures might be sufficient to control the infection in cases with no substantial preoperative infection. Indeed, successful regenerative outcomes have been reported without the need for inter-appointment medicaments using NaOCl alone or NaOCl followed by CHX for disinfection.

In conclusion, the results of this Example indicated that at least 1 mg/mL of DAP in a methylcellulose vehicle system is required to exert a significant antibiofilm effect that can eliminate substantial amount of three-week old E. faecalis biofilm. Furthermore, the 5-minute biofilm exposure to 1.5% NaOCl or 2% CHX irrigants provided an antibiofilm effect that was similar to a one week exposure to 500 mg/mL DAP or Ca(OH)₂ medicaments.

### Example 3

In this Example, the antibiofilm effect of antibiotic gel formulations of the present disclosure loaded into a vehicle system (e.g., methylcellulose) were analyzed against clinical isolates obtained from necrotic immature and mature teeth indicated for endodontic regeneration or routine endodontic treatment, respectively.

### Materials and Methods

Dentin Sample Preparation

Unidentified intact human teeth were collected, stored in 0.1% thymol solution at 4° C., and used within 6 months after obtaining local Institutional Review Board approval. A standardized radicular dentin sample (4×4×1 mm³) was obtained from each root using a slow diamond saw (IsoMet, Buehler, Lake Bluff, Ill.) under continuous distilled water irrigation. The pulpal side of each specimen was wet-finished with silicon carbide abrasive papers (500-2400 grit, Struers, Cleveland, Ohio). The polished samples were then sonicated in deionized water, 1.5% NaOCl and EDTA, washed with sterile water, wrapped individually with moist cotton pellets, placed in Whirl-pak bags (Sigma-Aldrich, St Louis, Mo.), gas sterilized with ethylene oxide, and stored at 4° C. until used.

Collection of Clinical Isolates

Two clinical mixed species biofilms were obtained during root canal treatment procedure of an immature tooth with a necrotic pulp that was indicated for endodontic regeneration treatment, as well as a mature tooth with a necrotic pulp that was indicated for conventional root canal therapy (IRB #1510640949). Each tooth was isolated with a rubber dam. Both tooth and rubber dam were then cleansed with 3% hydrogen peroxide solution and disinfected with 6% sodium hypochlorite solution. The coronal root canal access was performed with the use of sterile round burs. The pulp chamber was then disinfected using a swab soaked in 6% sodium hypochlorite solution. This solution was then inactivated with sterile 5% sodium thiosulfate. Samples were collected from the infected root canal by means of a #15 file with the handle cut off. The file will be introduced 1 mm short of the apical foramen and a filing motion was used for 30 seconds. 3 sterile paper points were inserted into the root canal at the same working length and were left inside for 1 minute in order to wick the tissue fluid. Both the file and paper points were placed into 2 mL of BHI-YE, vortexed to elute the bacteria, grown anaerobically at 37° C. for 48 hours and frozen at −80° C. until use.

Biofilm Growth on Dentin Specimens

The dentin specimens were sterilized in ethylene oxide and each specimen placed inside one well of a sterile 96 well plate with the pulp surface facing outward. 190 μl of fresh BHI-YE growth media and 10 μl of the clinically isolated multispecies biofilm from a mature tooth were added to ten of the wells in each experimental group and incubated anaerobically for three weeks at 37° C. The remaining ten wells of each experimental group were inoculated with 190 μl of fresh BHI-YE growth media and 10 μl of the clinically isolated bacterial culture from an immature tooth, and incubated anaerobically for three weeks at 37° C. Media were replaced every week during the incubation period. After that, infected dentin samples were treated with one of the experimental treatment groups.

Treatment of Infected Dentin

The dentin specimens were treated with 200 μL of the following: Group 1—5 mg/mL of DAP, Group 2—1 mg/mL of DAP, Group 3—Ca(OH)₂, Group 4—aqueous methyl cellulose (placebo), Group 5—no treatment, Group 6—BHI-YE without bacterial culture. All treatments were performed at 37° C. and 100% humidity, for a total treatment time of one week.

Biofilm Disruption Assays

Each dentin specimen was gently washed twice with sterile saline to remove the experimental paste and transferred to a new plastic test tube containing 200 μl of sterile saline. The tubes were sonicated for 20 seconds and vortexed for 30 seconds to detach biofilm cells. The detached biofilm cells were diluted and spirally plated on blood agar plates (CDC, BioMerieux). The plates were then incubated for 24 hours in 5% CO₂ at 37° C. and the number of CFUs/mL was determined by using an automated colony counter (Synbiosis, Inc., Frederick, Md.).

Statistical Analyses

Wilcoxon Rank Sum tests were used to compare bacteria results between biofilms from immature and mature teeth for each treatment, and for comparisons between each pair of treatments for biofilms from immature and mature teeth. Pair-wise comparisons were made using the Sidak method to control the overall significance level at 5% for each set of comparisons.

Results

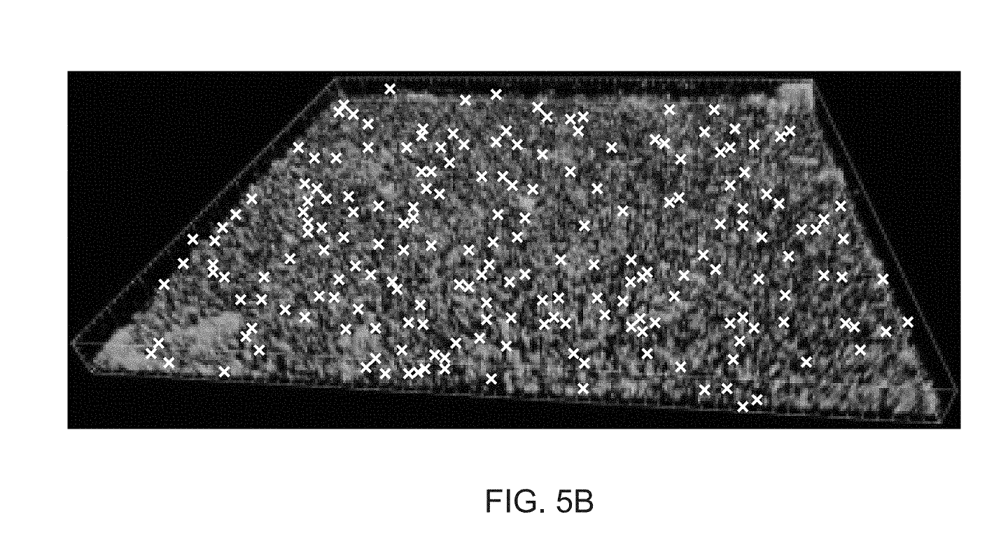

Groups treated with 1 mg/ml of DAP and 5 mg/mL of and Ca(OH)₂ demonstrated significant and substantial antibiofilm effects in comparison to untreated control groups or groups treated with placebo paste (FIG. 7). Furthermore, no significant differences were found among groups treated with 1 mg/ml of DAP, 5 mg/mL of Ca(OH)₂. These results indicate the ability of the innovative antibiotic gel formulations of the present disclosure to cause more than 99.9% reduction in bacterial biofilms formed from clinical isolates obtained from mature and immature necrotic teeth.

### Example 4

In this Example, the residual antibacterial effect in radicular dentin treated for one or four weeks with different dilutions of the antibiotic gel formulations of the present disclosure was analyzed.

### Materials and Methods

Extracted human teeth were prepared into 4×4 mm radicular dentin specimens and randomly assigned to 2 treatment times; treatment for 1 week or treatment for 4 weeks. Each time period includes 6 treatment groups (n=9 per group): G1—treated with 500 mg/mL of DAP without methylcellulose; G2—treated with 50 mg/mL DAP in paste form (i.e., with methylcellulose); G3—treated with 5 mg/mL DAP in paste form; G4—treated with 1 mg/mL DAP in paste form; G5—treated with Ca(OH)₂; and G6—methylcellulose paste without DAP.

After treatment, samples were placed in phosphate buffered saline (PBS) for three weeks. Samples were then infected with cultured E. faecalis and incubated in anaerobic conditions for three weeks to allow mature biofilm formation. After which, the dentin samples were rinsed and biofilms detached. The detached biofilm cells were then diluted and spirally plated for enumeration on blood agar plates. The plates were incubated for 24 hours in 5% CO₂ at 37° C. and the number of CFUs/mL determined using an automated colony counter.

### Results

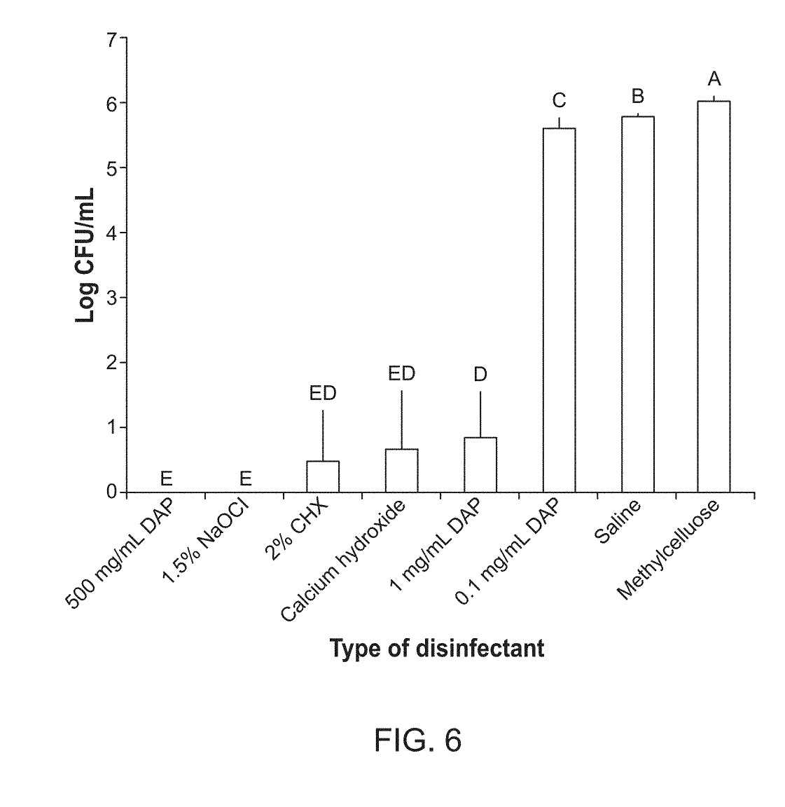

FIG. 8 demonstrates that 500 and 50 mg/mL of DAP showed significant residual antibiofilm effect for three weeks after only one week of application. Further, 500, 50, and 5 mg/mL of DAP caused complete eradication of three week old bacterial biofilm when they were applied for 4 weeks. The commercial available Ca(OH)₂ medicament did not show residual antibacterial effect even after application for 4 weeks.

### Conclusion

5 mg/mL of DAP in a methylcellulose system demonstrated the ability to completely eradicate three-week old bacterial biofilm. Furthermore, 5 mg/mL of DAP showed superior residual antibacterial effect in comparison to commercially available Ca(OH)₂ intracanal medicament.

### Example 5

In this Example, the residual antibacterial effects of dentin pretreated with the antibiotic gel formulations of the present disclosure were explored against two clinical isolates from mature and immature necrotic teeth.

### Materials and Methods

Experimental Groups

A total of 120 dentin specimens were prepared as described earlier in Example 3 and randomly assigned into 6 experimental groups (n=20 per group). Each experimental group included 10 dentin samples inoculated with the clinical isolates from mature necrotic tooth and the remaining 10 samples were inoculated with clinical isolates from immature necrotic tooth.

Human Dentin Specimens Treatment

This Example focused on the residual antibacterial effect of DAP. Thus, it was assumed that the dentin had already been disinfected through irrigation and medicament, and there were no initial viable bacterial biofilm before medicament application. The specimens were sterilized in ethylene oxide. There were 20 dentin specimens for each experimental group: Group 1—1 mg/mL of DAP, Group 2—5 mg/mL of DAP, Group 3—Ca(OH)₂, Group 4—Sterile water and methylcellulose used as a placebo paste, Group 5—no treatment with bacteria, Group 6—no treatment/no bacteria. These dentin samples were treated with 200 μL of the aforementioned treatment groups for 1 week at 37° C. and 100% humidity to prevent dehydration. After the 1-week period, the specimens were irrigated for one minute with 5 ml of sterile saline followed by irrigation with 5 ml of 17% EDTA for 5 minutes. Samples were then kept independently in phosphate buffered saline (PBS) for 3 weeks.

Bacterial Strains and Media

Anaerobic blood agar plates (CDC, BioMerieux, Durham, N.C.) were used to initially grow and maintain the separate clinically isolated biofilms. An infusion broth of brain heart broth supplemented with 5 g/L yeast extract (BHI-YE) was used to grow each bacterial biofilm at 37° C. in an anaerobic environment using gas generating sachets (GasPak EZ, Becton, Dickinson and Company, Franklin Lakes, N.J.) to produce the required environment. The two bacterial sample collections were performed during the root canal treatment as described earlier in Example 3.

Bacterial Growth on Root Specimens

Root specimens were removed from PBS and placed individually inside a well of a sterile 96-well plate with the pulpal surface facing outward. Then, 190 μl of fresh BHI-GE growth media and 2 days of clinically isolated bacterial species from an adult mature necrotic tooth were added to 10 of the wells in each experimental group and incubated anaerobically at 37° C. for 3 weeks before performing the antibacterial testing. The remaining 10 wells of each experimental group were inoculated with 190 μl of fresh BHI-GE growth media and 10 μl of a 48-hour culture of the clinically isolated bacterial sample from an immature tooth with pulpal necrosis. These wells were incubated anaerobically at 37° C. for 3 weeks before performing the antibacterial testing. Culture media were replaced every week during incubation.

Biofilm Disruption Assays

After bacterial biofilms had been allowed to grow for 3 weeks, each dentin sample was individually transferred into a fresh 200 μl tube of sterile saline. Tubes were sonicated for 20 seconds and vortexed for 30 seconds to detach biofilm cells. Biofilms that have been removed were diluted (1:10 and 1:1,000) and spirally plated on blood agar plates (CDC, BioMerieux). Bacterial plates were then incubated at 37° C. for 24 hours in 5% CO₂. The number of CFUs/mL was determined by using an automated colony counter (Synbiosis, Inc., Frederick, Md.).

Statistical Methods

The effects of treatment and type of biofilm on bacteria counts were analyzed using two-way ANOVA. Pair-wise comparisons among the treatment combinations were made using the Sidak method to control the overall significance level at 5% for each set of comparisons.

### Results

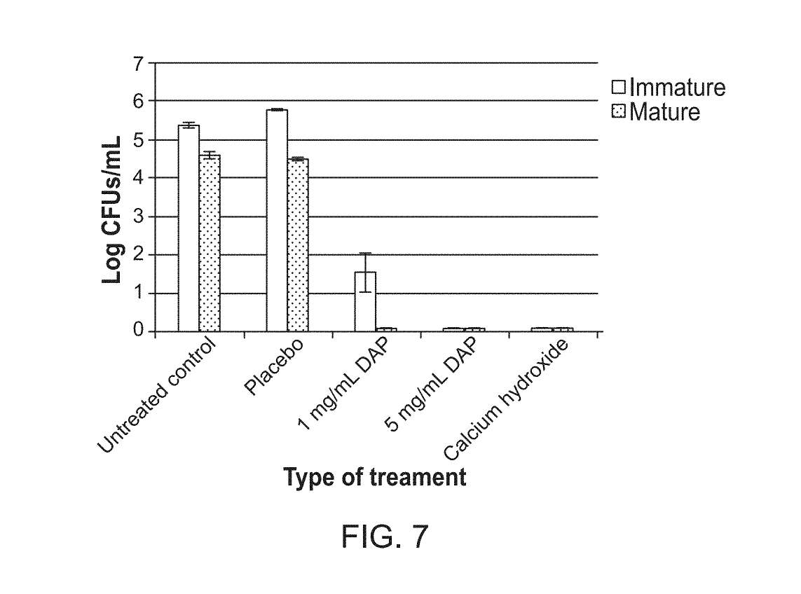

Bacteria growth were lower for biofilms isolated from mature tooth than that of isolated immature tooth in experimental groups treated with 1 or 5 mg/ml of antibiotic formulations (≤0.005), but no difference was found between mature and immature biofilms for control, placebo, or (Ca(OH)₂ treatments (FIG. 9). Dentin pretreated with Ca(OH)₂ or placebo paste did not demonstrate any significant residual antibacterial effects regardless of the source of biofilm. Dentin pretreated with 1 mg/ml of antibiotic formulation demonstrated significant residual antibacterial effects in comparison to untreated control, placebo and Ca(OH)₂ treated dentin only in biofilms isolated from mature teeth. The residual antibacterial effects of dentin pretreated with 5 mg/ml of antibiotic formulation were significantly higher than all other groups regardless of the source of biofilm. These data indicate that the antibiotic gel formulation of the present disclosure offers significantly better residual antibacterial effects against clinical isolates in comparison to the commercially used intracranal medicaments (Ca(OH)₂). Furthermore, these data also confirm the ability of the antibiotic gel formulation of the present disclosure to provide extended antibacterial effects.

### Example 6

In this Example, a clinical case of an immature necrotic tooth treated with an endodontic regeneration approach using 10 mg/ml of the antibiotic gel formulation of the present disclosure as intracanal medicament was demonstrated.

First Visit

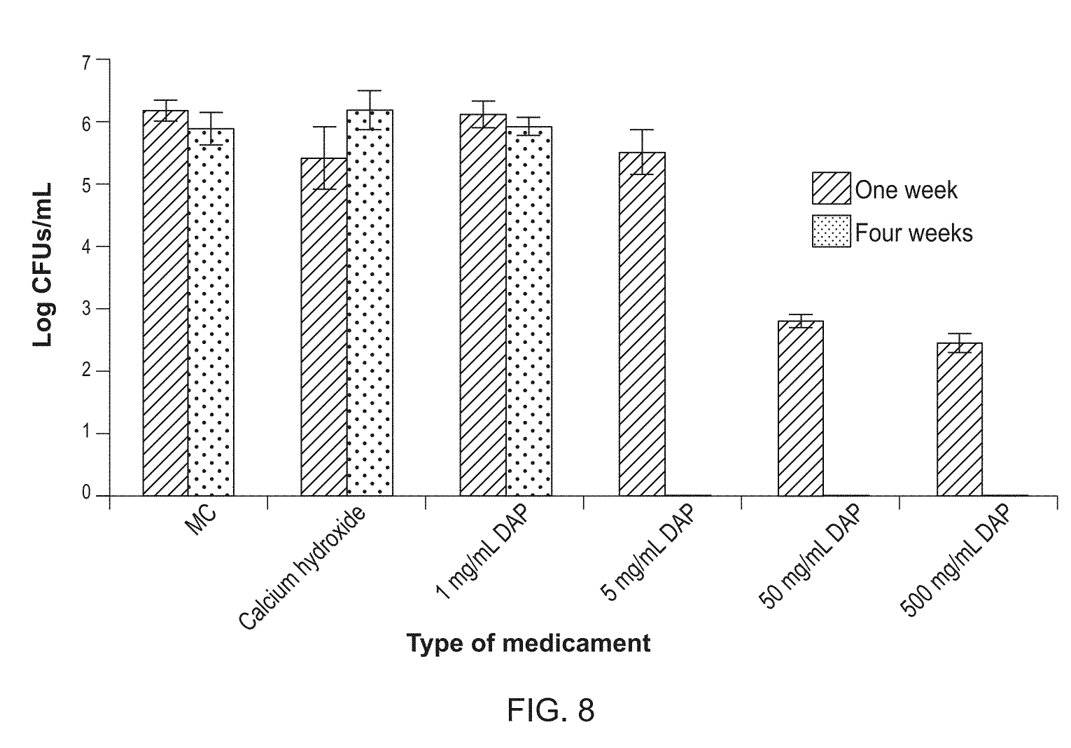

Necrotic immature permanent upper incisor with periapical abscess (FIG. 10A) and sinus tract (FIG. 10B) was treated according to the recent guidelines of American Association of Endodontists. That is, the pulp chamber was accessed, canal length was established, and pus was drained from the root canal using small capillary tubes connected to high speed suction. The canal was rinsed with 20 ml of 1.5% sodium hypochlorite, followed by 20 ml of sterile saline, and dried with paper points. A 10 mg/ml paste of the antibiotic gel formulation of the present disclosure was prepared as described in Example 1 and delivered into the canal using a syringe application in the delivery system of the present disclosure.

Second Visit

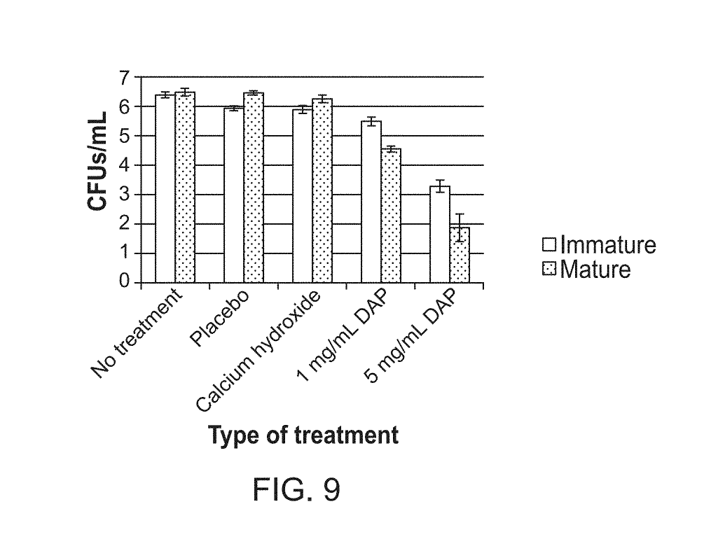

Seven weeks after the initial visit, no clinical signs and symptoms were observed. Furthermore, there was complete healing of the periapical sinus tract (FIG. 11). The canal was accessed, irrigated with 17% EDTA, and dried with paper points. A size 60 endodontic file was used to lacerate the apical papilla and induce bleeding into the canal up to the cemento-enamel junction. A small piece of CollaTape was placed over the clot and 3 mm of white MTA was applied to seal the access opening followed by composite resin permanent restoration.

Third Visit

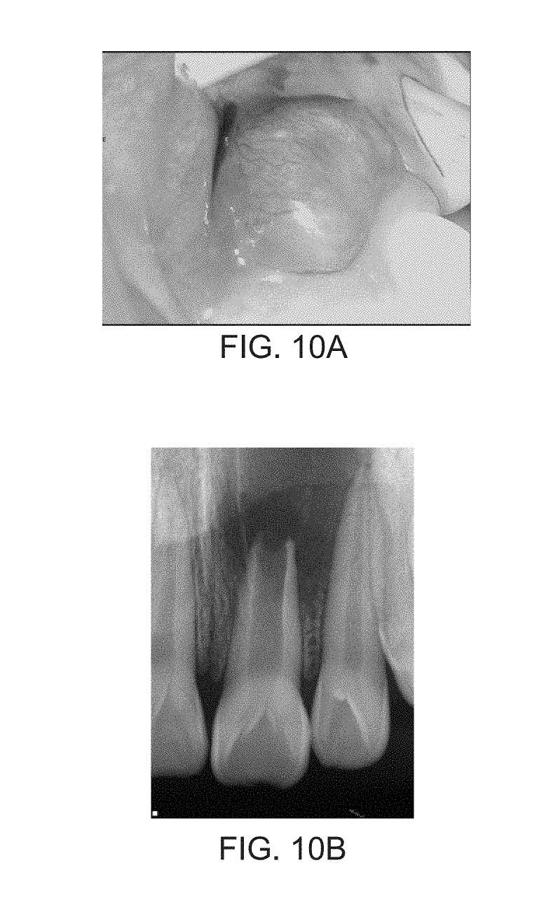

Five months after the initial visit. No clinical signs and symptoms were reported. Furthermore, a radiographic evidence of periapical healing was clear (FIG. 12). The periapical radiograph shows 30-50% reduction in the size of the periapical lesion (abscess), which indicates a bony healing.

Fourth Visit

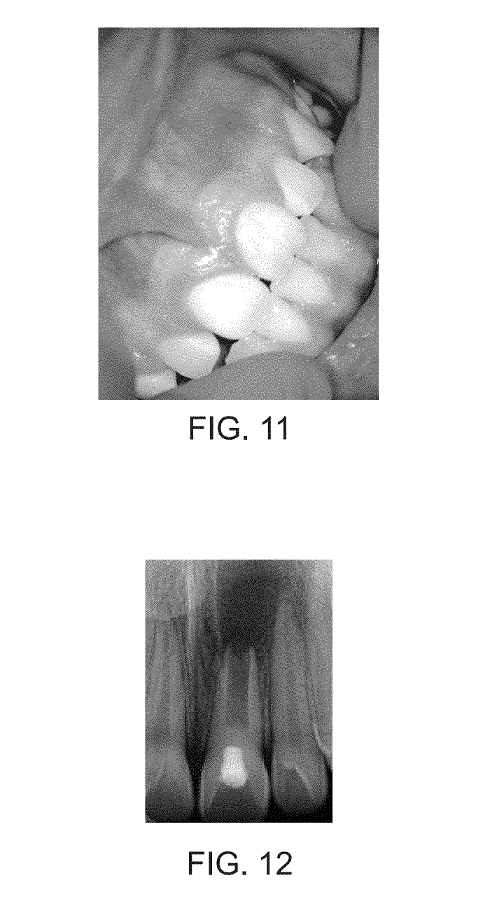

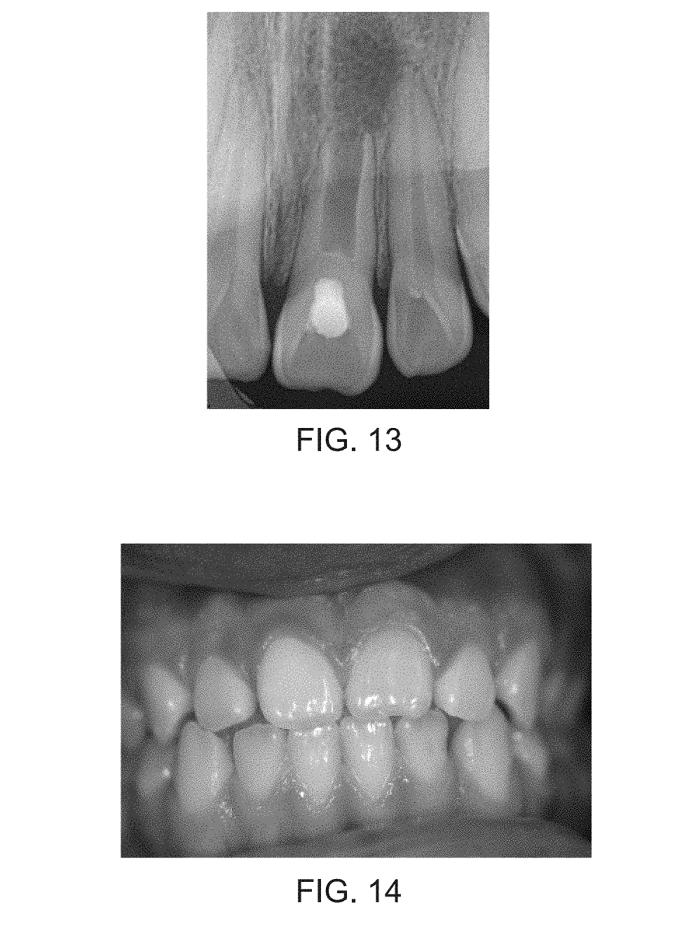

One year after the initial visit, no clinical signs and symptoms were reported. Furthermore, a radiographic evidence of periapical healing was clear (FIG. 13). The periapical radiograph showed clear bony healing and resolution of the periapical radiolucency. Furthermore, no crown discoloration was observed clinically (FIG. 14).

### Example 7

In this example, the antibiofilm effects of radiopaque antibiotic gel formulations of the present disclosure loaded into a vehicle system (e.g., methylcellulose) were analyzed against a clinical isolate obtained from necrotic immature tooth that was indicated for endodontic regeneration treatment.

### Materials and Methods

Preparation of Radiopaque Antimicrobial Gel Formulations

Various concentrations of radiopaque DAP (1, 10 and 20 mg/mL) were prepared as follows: 500, 250 and 25 mg of equal portions of metronidazole and ciprofloxacin USP grade powders (Champs Pharmacy, San Antonio, Tex.) were dissolved in 25 mL of sterile water, respectively. Then, 8.75 grams of barium sulfate powder (Reagent plus, Sigma-Aldrich) was blended gradually into each antibiotic solution using a lab mixer. Finally, 1.75 grams of methylcellulose powder (Methocel 60 HG, Sigma-Aldrich) was gradually incorporated into each mixture under vigorous stirring at room temperature to obtain homogenous gel formulations with 1, 10 and 20 mg/mL concentrations of DAP. A placebo radiopaque methylcellulose paste with no DAP was also prepared. Additionally, a commercially available Ca(OH)₂ (UltraCal XS; Ultradent, South Jordan, Utah) was also used.

Preparation of Dentin Samples and Collection of the Clinical Isolate

In this Example, the preparation and sterilization of dentin samples was performed as described earlier in Example 3. Furthermore, the bacterial clinical isolate was obtained from the root canal of a necrotic immature tooth that was indicated for endodontic regenerative procedure as described earlier in Example 3.

Infection of Dentin Samples

The sterilized dentin samples were placed inside one well of a sterile 96-well plate with the pulp surface facing outward. Then, 190 μl of fresh BHI-YE growth media and 10 μl of the clinically isolated biofilm was added to each of the dentin samples and incubated anaerobically for three weeks at 37° C. with weekly replacement of culture media. After that, infected dentin samples were treated with one of the experimental treatment groups.

Treatment of Infected Dentin

The dentin samples were randomly assigned into 7 experimental groups (n=10 per group) and treated with 200 μL of the following: Group 1—1 mg/mL of radiopaque DAP, Group 2—10 mg/mL of radiopaque DAP, Group 3—20 mg/mL of radiopaque DAP, Group 4—Ca(OH)₂, Group 5—aqueous radiopaque methylcellulose paste (placebo), Group 6—no treatment, Group 7—BHI-YE without bacterial culture. All treatments were done at 37° C. and 100% humidity, for a total treatment time of one week.

Biofilm Disruption Assays

After one week, the samples were gently washed for 1 minute with sterile saline to remove the medicaments and biofilm disruption assays were then performed as previously described Example 3.

Results

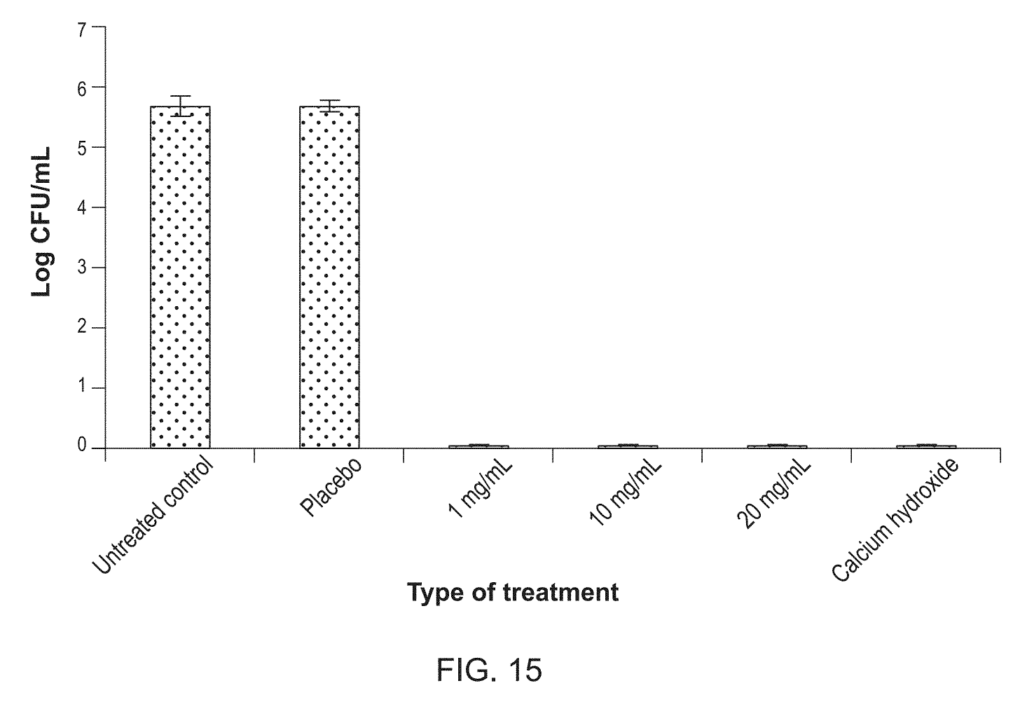

The three concentrations of radiopaque antibiotic gel formulation, as well as calcium hydroxide, demonstrated the ability to eradicate bacterial biofilms (FIG. 15). However, the radiopaque placebo paste did not show any antibacterial effect in comparison to the untreated dentin samples. These data clearly support the ability of the radiopaque antibiotic gel formulations of the present disclosure to exert antibacterial effects.

In view of the above, it will be seen that the several advantages of the disclosure are achieved and other advantageous results attained. As various changes could be made in the above formulations, delivery systems and methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A delivery system for delivering an antibiotic gel formulation into a root canal, the delivery system comprising: an applicator and an antibiotic gel formulation, the antibiotic gel formulation comprising an antibiotic selected from the group consisting of metronidazole, ciprofloxin, and combinations thereof, and a thickening agent.

2. The delivery system of claim 1 wherein the applicator is a syringe comprising a tip cap and a delivery tip shaped to fit into a root canal.

3. The delivery system of claim 2 wherein the delivery tip comprises a length of from about 10 mm to about 30 mm.

4. The delivery system of claim 2 wherein the delivery tip comprises a diameter of from about 0.25 mm to about 1.25 mm.

5. The delivery system of claim 2 wherein the delivery tip comprises a diameter of from about 0.5 mm to about 1.0 mm.

6. The delivery system of claim 2 wherein the tip cap comprises a stopper.

7. The delivery system of claim 1 wherein the antibiotic consists of metronidazole and ciprofloxin in a ratio of metronidazole to ciprofloxin of about 1:1.

8. The delivery system of claim 1 wherein the antibiotic gel formulation comprises from about 1 mg/ml to about 50 mg/ml of the antibiotic.

9. The delivery system of claim 1 wherein the antibiotic gel formulation comprises from about 2 mg/ml to about 10 mg/ml of the antibiotic.

10. The delivery system of claim 1 wherein the thickening agent comprises methylcellulose.

11. The delivery system of claim 10 wherein the antibiotic gel formulation comprises from about 60 to about 110 mg/ml methylcellulose.

12. The delivery system of claim 1 wherein the antibiotic gel formulation further comprises an imaging agent.

13. The delivery system of claim 12 wherein the antibiotic gel formulation further comprises barium sulfate.

14. A method of applying an antibiotic gel formulation to a root canal, the method comprising:
applying the antibiotic gel formulation with a syringe comprising a tip cap and a delivery tip shaped to fit into a root canal, the angled delivery tip comprising a diameter of from about 0.25 mm to about 1.25 mm; and
allowing the antibiotic formulation to remain in the root canal for a period of from about 1 week to about 2 months.

15. The method of claim 14 wherein the delivery tip comprises a diameter of from about 0.5 mm to about 1.0 mm.

16. The method of claim 14 wherein the delivery tip comprises a length of from about 10 mm to about 30 mm.

17. The method of claim 14 wherein the tip cap comprises a stopper.

18. The method of claim 14 comprising allowing the antibiotic formulation to remain in the root canal for a period of from about 2 weeks to about 4 weeks.

19. The method of claim 14 further comprising removing the antibiotic formulation from the root canal by rinsing the canal with ethylenediaminetetraacetic acid (EDTA).

20. The method of claim 14 wherein the antibiotic gel formulation comprises an antibiotic selected from the group consisting of metronidazole, ciprofloxin, and combinations, and a thickening agent.
